(19) United States
(12) Patent Application Publication
(10) Pub. No.: US 2024/0174588 A1
(43) Pub. Date: May 30, 2024

(54) METHOD FOR PREPARING CHIRAL ALKYL COMPOUNDS BY ASYMMETRIC HYDROGENATION OF OLEFINS CATALYZED BY IRON COMPLEX

(71) Applicant: Zhejiang University, Hangzhou (CN)

(72) Inventors: Zhan Lu, Hangzhou (CN); Peng Lu, Hangzhou (CN); Xiang Ren, Hangzhou (CN)

(21) Appl. No.: 18/210,771

(22) Filed: Jun. 16, 2023

Related U.S. Application Data
(63) Continuation of application No. PCT/CN2021/130103, filed on Nov. 11, 2021.

(30) Foreign Application Priority Data
Jan. 22, 2021 (CN) 202110092710.1

(51) Int. Cl. C07C 41/20; B01J 31/12

(52) U.S. Cl. CPC C07C 41/20; B01J 31/12; C07B 2200/09; C07C 2531/12

(57) ABSTRACT

Provided in the present invention is a method for preparing chiral alkyl compounds by the asymmetric hydrogenation reaction of iron complex catalysts catalysing olefins: using the disubstituted olefin shown in formula I as a raw material, atmospheric hydrogen as a hydrogen source, FeX2-8-OIQ complex as a catalyst, and a silane compound and acetonitrile as cocatalysts, and reacting for 12-24 hours under the action of a reducing agent to prepare the chiral alkyl compound shown in formula II. The method of the present invention has mild reaction conditions, simple operation, and high atom economy. In addition, the reaction does not require the addition of any other toxic transition metal (such as ruthenium, rhodium, and palladium), and has great practical application value in the synthesis of drugs and materials. The conversion rate of the reaction is also good, generally reaching >99%, and the enantioselectivity is also high, generally 70-99%.

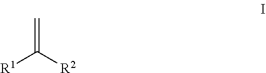

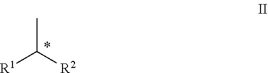

### TECHNICAL FIELD

The method refers to preparing chiral alkyl compounds by asymmetric hydrogenation of 1,1-disubstituted olefins catalyzed by an iminoquinoxazoline (8-OIQ) iron complex catalyst. In particular, this method could be used to prepare optically active chiral drug molecular intermediates.

### BACKGROUND

Chirality is a basic attribute of nature. The “thalidomide event” has made people gradually realized the importance of chirality. Asymmetric hydrogenation of olefins is one of the most effective methods to obtain chiral compounds due to its atom-economy, simple operation, high activation and environmental-friendly. It has been widely used in the synthesis of chiral drugs, natural products, and industrial production of pesticide molecules. Knowles and Noyori won the Nobel Prize in Chemistry. [a) Knowles, W. S. Angew. Chem. Int. Ed. 2002, 41, 1998; b) Noyori, R. Angew. Chem. Int. Ed. 2002, 41, 2008.]

The core of the asymmetric hydrogenation of olefins is the design of metals and chiral ligands. The noble metals, such as rhodium, ruthenium and iridium play an dominant role in this field. The chiral ligands are mainly focus on bidentate phosphine containing ligands, such as DIPAMA, BINAP and Duphos. [a) Knowles, W. S.; Sabacaky, M. J.; et al. J. Am. Chem. Soc. 1975, 97, 2567; b) Miyashita, A.; Yasuda, A.; Takaya, A.; et al. J. Am. Chem. Soc. 1980, 102, 7932; c) Noyori, R.; Ohta, M., Hsiao, Y.; et al. J. Am. Chem. Soc. 1986, 108, 7117; d) Burk, M. J. Acc. Chem. Res. 2000, 33, 363; e) Xie, J. H.; Wang, L. X.; Fu, Y; et al. J. Am. Chem. Soc. 2003, 125, 4404; f) Tolstoy, P.; Engman, M.; Paptchikhine, A.; Bergquist, J.; Church, T. L.; Leung, A. W. -M.; Andersson, P. G.; J. Am. Chem. Soc. 2009, 131, 8855.; g) Kaiser, S.; Smidt, S. P.; Pfaltz, A. Angew. Chem. Int. Ed. 2006, 45, 5194.; h) Biosca, M.; Magre, M.; Pamies, O.; Diéguez, M.; ACS Catal. 2018, 8, 10316.; i) Perry, M. C.; Cui, X. H.; Powell, M. T.; Hou, D. -R.; Reibenspies, J. H.; Burgess, K. J. Am. Chem. Soc. 2003, 125, 113.]. However, the high cost, low earth abundance, and environmental impact associated with these noble elements inspires the search for catalysts with less toxic, more cost-effective, earth-abundant metals, such as Fe, and Co. Moreover, the potential unique features in 3d metal catalysis, such as a single-electron process, smaller d orbitals, multiple oxidation and spin states might provide new opportunities to solve the challenges of noble metal catalytic systems. Among them, iron is the second largest metal element in the earth's crust, and it has good biological compatibility. So far, the iron-catalyzed asymmetric hydrogenation of alkenes is not be realized. Therefore, it is very important to develop new ligands and iron catalysts for asymmetric hydrogenation of olefins. [a) Bart, S. C.; Lobkovsky, E.; Chirik, P. J. J. Am. Chem. Soc. 2004, 126, 13794-13807; b) Hoyt, J. M.; Shevlin, M.; Margulieux, G. W.; Krska, S. W.; Tudge, M. T.; Chirik, P. J. Organometallics 2014, 33, 5781-5790. c) Chirik, P. J. Acc. Chem. Res. 2015, 48, 1687. d) Guo, N.; Hu, M. -Y.; Feng, Y.; Zhu, S. -F. Org. Chem. Front., 2015, 2, 692-696; e) Frank, D. J.; Guiet, L.; Käslin, A.; Murphy, E.; Thomas, S. P. RSC Adv. 2013, 3, 25698. f) E. J. Daida and J. C. Peters, Inorg. Chem., 2004, 43, 7474. g) Sunada, Y; Ogushi, H.; Yamamoto, T.; Uto, S.; Sawano, M.; Tahara, A.; Tanaka, H.; Shiota, Y; Yoshizawa, K.; Nagashima, H. J. Am. Chem. Soc., 2018, 140, 4119-4134.]. According to literature research, the iron-catalyzed asymmetric hydrogenation of olefins has the following challenges. First, iron has variable valence (−2 to +5), so it is challenging to design appropriate ligands to stabilize the metal valence of iron. Secondly, the iron is easy to dissociate with ligand after the catalyst was activated by activating reagent, which leads to the background reaction and get racemized product. [Hoyt, J. M.; Shevlin, M.; Margulieux, G. W.; Krska, S. W.; Tudge, M. T.; Chirik, P. J. Organometallics 2014, 33, 5781.]

In 2016, the Lu group developed an oxazoline iminopyridine (OIP) coordinated cobalt-catalyzed asymmetric hydrogenation of 1,1-disubstituted alkenes. [Chen, J. -H.; Chen, C. -H.; Ji, C. -L.; Lu, Z. Org. Lett. 2016, 18, 1594.]. Subsequently, the OIP coordinated iron catalyst was used to catalyze the asymmetric hydrogenation of 1,1-disubstituted olefins. Unfortunately, the product was obtained with low yield and enantiopure excess. Therefore, it is of great significance to develop new iron catalysts for asymmetric hydrogenation of simple olefins.

### BRIEF SUMMARY

The invention includes the asymmetric hydrogenation of minimally functionalized 1,1-disubstituted alkenes using the oxazoline iminoquinoline ligand (8-OIQ) iron complex, A series of substrates with various functional groups could smoothly transfer to products with good conversion and enantioselectivity. In addition, the catalyst could hydrogenate the 1,1-disubstituted olefins sterically specifically and the hindered trisubstituted C═C double bond was maintained. Through this invention, we can also easily synthesize useful pharmaceutical molecular intermediates.

The invention is realized by the following technical program:

A method for preparing chiral alkyl compounds by asymmetric hydrogenation of olefins catalyzed by iron complex catalyst, the method is as follows: a disubstituted olefin shown in Formula I is used as a raw material, atmospheric hydrogen is used as hydrogen source, FeX₂-8-OIQ complex is used as catalyst, hydrosilane and acetonitrile are used as co catalysts, and a chiral alkyl compound shown in Formula II is prepared by reaction for 12-24 hours under the activation of a reducing agent;

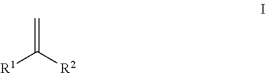

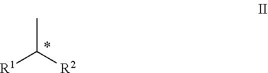

in Formula II, * represents a chiral carbon atom.

The reaction formula of the invention could be expressed as follows:

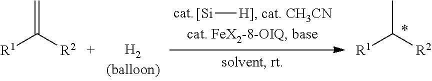

In Formula I or Formula II, R¹ is optionally selected from C₂˜C₈ alkyl, naphthyl, a group shown in Formula III, or a N and O containing heterocyclic aryl group of C₄˜C₁₀;

in R¹, the H on C₂˜C₈ alkyl is not substituted or substituted by at least one substituent A, and the substituent A is phenyl, naphthyl, heterocyclic aryl or substituted phenyl; heterocyclic aryl is indolyl, pyridinyl, pyrrolyl, thiophenyl or furanyl; substituted phenyl is the phenyl group in which H on phenyl is replaced by at least one substituent B, and the substituent B is C₁˜C₃ alkylp, C₁˜C₃ alkoxy, halogen or C₁˜C₃ alkylthio;
in R¹, the N and O containing heterocyclic aryl group of C₄˜C₁₀ is pyridinyl, pyrrolyl, indolyl, benzodioxazolyl, benzoxazolyl or furanyl, preferably pyridinyl, indolyl or benzodioxazolyl;
in R¹, the H on naphthyl and the N and O containing heterocyclic aryl group of C₄˜C₁₀ are not substituted or substituted by at least one substituent C, and the substituent C is C₁˜C₃ alkyl or C₁˜C₃ alkoxy;

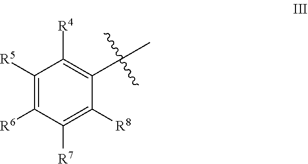

in R¹, among the groups shown in Formula III, R⁴, R⁵, R⁶, R⁷, R⁸ are selected from any one of H, halogen, C₁˜C₃ alkyl, C₁˜C₃ alkoxy, benzyloxy, C₁˜C₃ alkylthio, tert butyl dimethyl siloxy, trifluoromethyl, dimethylamino, pinacol borate, d-borneoxy, citronellol oxy, menthol oxy or geraniol oxy, and when R⁴, R⁵, R⁶, R⁷, R⁸ are all H, Formula III is phenyl; and halogen is F or Cl.

In Formula I or Formula II, R² is optionally selected from C₁˜C₈ alkyl, C₂˜C₈ alkenyl, phenyl or benzyl; the H on C₁˜C₈ alkyl and C₂˜C₈ alkenyl are not substituted or substituted by at least one substituent D, which is phenyl, substituted phenyl, C₁˜C₃ amino or 1,3-dioxolacyl.

Or in Formula I or Formula II, R¹ and R² are connected into a ring to form C₉˜C₁₂ benzocycloalkyl; H on C₉˜C₁₂ benzocycloalkyl is not substituted or substituted by at least one substituent E, wherein the substituent E is C₁˜C₃ alkyl, C₁˜C₃ alkoxy or halogen; preferably, R¹ and R² are linked to form benzocyclohexane.

R¹ and R² are different substituents.

Further, R¹ is preferably alkyl, naphthyl, 6-methoxynaphthalyl, pyridinyl, 2-methoxypyridyl, indolyl, N-methylindolyl, benzodioxazolyl or the group shown in Formula III.

In R¹, when R¹ is C₂˜C₈ alkyl, H on C₂˜C₈ alkyl is replaced by a substituent A. R¹ can be expressed as R^{A}—(CH₂)ₙ—, where n is an integer of 2 to 8. R^{A} is a substituent A on the carbon chain, and R^{A} is preferably phenyl, naphthyl or p-methoxyphenyl.

In R¹, the group shown in Formula III is preferably phenyl or substituted phenyl with 1-2 substituents, and the substituent on the substituted phenyl is preferably halogen, C₁˜C₂ alkyl, C₁˜C₃ alkoxy, benzyloxy, C₁˜C₃ alkylthio, tent-butyl dimethyl siloxy, trifluoromethyl, dimethylamino, pinacol borate, d-borneol, citronellol-oxyl, menthol-oxyl or geraniol-oxyl.

R² is preferably C₂˜C₆ alkyl, C₂˜C₆ alkenyl, phenyl or benzyl; H on C₂˜C₆ alkyl is not substituted or substituted by substituent D, R² is the C₂˜C₆ alkyl, and when H on the alkyl is substituted by substituent D, R² can be expressed as R^{D}—(CH₂)ₘ—, m is an integer of 2 to 6, and the substituent D is preferably phenyl, C₁˜C₃ amino, 4-methoxyphenyl or 1,3-dioxolacyl.

In the invention, the silane compound is benzene silane (PhSiH₃) or n-octadecyl silane (n-C₁₈H₃₇SiH₃).

As a further improvement, an organic solvent is added to the method of this invention, and the organic solvent is any one of benzene, carbon tetrachloride, toluene, tetrahydrofuran, ether, dichloromethane, acetonitrile, dioxane, petroleum ether, cyclohexane, n-hexane, ethyl acetate, chloroform, and dimethylformamide, preferably toluene.

The amount of the organic solvent is generally 1-10 mL/mmol based on the amount of the disubstituted olefin shown in Formula I.

The reducing agent of the invention is any one of sodium triethylborohydride, sodium tri-sec-butyl-borohydride, lithium triethylborohydride, sodium tert-butoxide, potassium tert-butoxide, lithium tert-butoxide, sodium tert-pentoxide, sodium ethoxide, sodium methoxide and potassium methoxide, preferably is sodium triethylborohydride, sodium tert-butoxide, sodium ethoxide or sodium methoxide, more preferably is sodium triethylborohydride.

The invention takes the atmospheric hydrogen as the hydrogen source and inserts a hydrogen balloon on the reaction bottle.

As a further improvement, the mole ratio of the disubstituted olefin shown in Formula I, FeX₂-8-OIQ complex, silane compound, acetonitrile and reducing agent of the invention is 1:0.00001-0.1:0.02-0.2:0.1-0.3:0.06-0.3, preferably 1:0.001-0.05:0.2: 0.2: 0.15.

As a further improvement, the reaction temperature of the invention is 0° C.˜room temperature.

As a further improvement, after the reaction of the invention is completed, the obtained crude product is treated to obtain the chiral alkyl compound shown in Formula II. The treatment method includes thin-layer chromatography, column chromatography or vacuum distillation, preferably column chromatography.

The catalyst FeX₂-8-OIQ complex (8-OIQ: 8-oxazoline iminoquinoline ligand) used in the invention is an optically pure compound shown in Formula IV or its enantiomer or racemate. In Formula IV, R⁹ is C₁˜C₁₂ alkyl which is unsubstituted or substituted by 1 or 2 C₁˜C₄ alkoxys, C₅˜C₁₂ cycloalkyl which is unsubstituted or substituted by 1-3 substituents a, or aryl a which is unsubstituted or substituted by 1-4 substituents b; the aryl a is benzyl, phenyl or naphthyl; the substituent a is C₁˜C₄ alkyl or C₁˜C₄ alkoxy; and the substituent b is C₁˜C₄ alkyl, C₁˜C₄ alkoxy, C₁˜C₄ fluoroalkyl, Cl˜C₄ fluoroalkoxy, F or Cl;

R¹⁰ is H, C₁ ˜C₁₂ alkyl which is unsubstituted or substituted by 1-2 C₁˜C₄ alkoxy, C₅˜C₁₂ cycloalkyl which is unsubstituted or substituted by 1-3 substituents a, or aryl b which is unsubstituted or substituted by 1-3 substituents b; the aryl b is phenyl or naphthyl; the substituent a is C₁˜C₄ alkyl or C₁˜C₄ alkoxy; and the substituent b is C₁˜C₄ alkyl, C₁˜C₄ alkoxy, C₁˜C₄ fluoroalkyl, C₁˜C₄ fluoroalkoxy, F or Cl;

R¹¹, R¹², R¹³, R¹⁴ and R¹⁵ are independently H, C₁˜C₁₂ alkyl, C₁˜C₄ fluoroalkoxy, F, Cl, nitro or C₅˜C₁₂ cycloalkyl which is unsubstituted or substituted by 1-3 substituents a;

R¹⁶ and R¹⁷ are independently H, C₁˜C₁₂ alkyl which is unsubstituted or substituted by 1-2 C₁˜C₄ alkoxy groups, C₅˜C₁₂ cycloalkyl which is unsubstituted or substituted by 1-3 substituents a, or aryl a which is unsubstituted or substituted by 1-3 substituents b;

R¹⁸ is a C₁˜C₁₂ alkyl group that is not substituted or substituted by 1-2 C₁˜C₄ alkoxy groups, a cycloalkyl group of C₅˜C₁₂ that is not substituted or substituted by 1-3 substituents a, or an aryl a that is not substituted or substituted by 1-3 substituents b;

In Formula IV, * represents a chiral carbon atom.

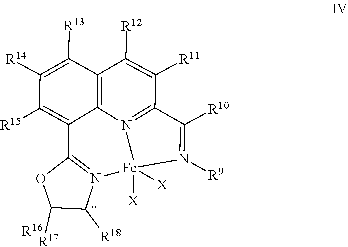

X is F, Cl, Br, I, OAc or CF₃SO₃, preferably Cl or Br.

Further, the catalyst FeX₂-8-OIQ complex is preferably a compound shown in Formula IV, wherein R¹¹, R¹², R¹³, R¹⁴, R¹⁵, R¹⁶ and R¹⁷ are preferably H; R¹⁰ is preferably C₁˜C₄ alkyl or diphenylmethylene; preferably, R⁹ is C₁˜C₄ alkyl, benzyl, phenyl or 2,6-diisopropylphenyl; preferably, R¹⁸ is C₁˜C₄ alkyl, benzyl or phenyl; X is Cl or Br.

Furthermore, the used chiral FeX₂-8-OIQ complex is preferably shown in Formula IV-1 or Formula IV-2

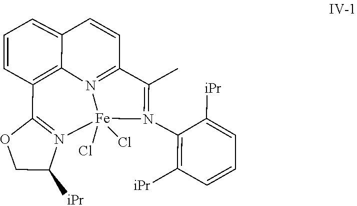

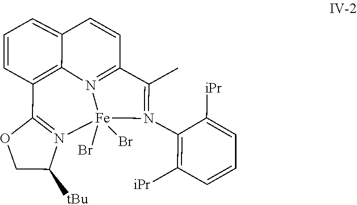

In the invention, the compound shown in Formula IV can be prepared by the following method:
under nitrogen protection, a chiral iminoquinoline oxazoline compound shown in formula (1) react with ferrous salt FeX₂ in an organic solvent for 1-10 hours to prepare the complex shown in formula IV; the organic solvent is tetrahydrofuran or 2-methyltetrahydrofuran;

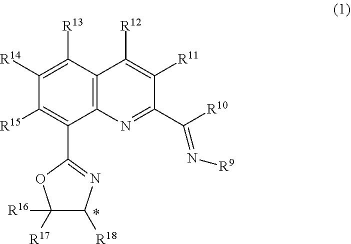

R⁹ ˜R¹⁸ and * in formula (1) are all as described previously.

The mole ratio of the chiral iminoquinoline oxazoline compound shown in formula (1) and ferrous salt FeX₂ is 2.2-0.9:1, preferably 1.1-0.9:1, more preferably 1.1-1:1.

The synthesis of metal compounds shown in Formula IV can be carried out at low or high temperatures, such as −20˜150° C., preferably at room temperature.

The chiral iminoquinoline oxazoline compounds shown in formula (1) can be prepared by the following method:
(a) a 2-acyl-8-bromoquinoline compound shown in formula (2) and an amine compound shown in formula (3) are subjected to condensation reaction under the action of a catalyst to obtain a compound shown in formula (4);
(b) under the protection of nitrogen, the compound shown in formula (4) and an oxazoline compound shown in formula (5) are subjected to coupling reaction under the catalysis of a transition metal inorganic salt, an organic phosphine ligand and an inorganic base to obtain the chiral iminoquinoline oxazoline compound shown in formula (1);

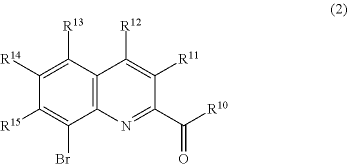

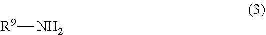

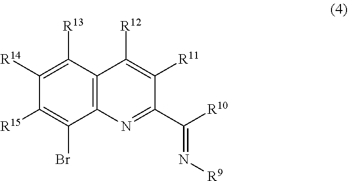

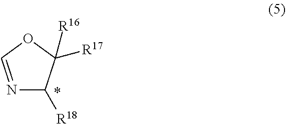

R⁹˜R¹⁸ and * are as previously described.

In step (a), the mole ratio of the 2-acyl-8-bromoquinoline compound shown in formula (2) and amine compound shown in formula (3) is 1:1-10, preferably 1:1-5, more preferably 1:1-2.

The step (a) is carried out under the action of catalyst. The catalyst is proton acid or molecular sieve, preferably p-toluenesulfonic acid. The amount of the catalyst material is 1-5mol % of the 2-acyl-8-bromoquinoline compound shown in formula (2).

The reaction solvent of step (a) is an organic solvent, preferably toluene, benzene or xylene, more preferably toluene.

The reaction in step (a) needs to be heated to reflux, a water separator is used to separate water during reaction, and reaction time is 15-30 hours.

Step (b) is a coupling reaction catalyzed by the inorganic salt of transition metal Ru, Rh, Pd, Ir, organic phosphine ligand and inorganic base.

The step (b) is carried out under the catalysis of the transition metal inorganic salt, organic phosphine ligand and inorganic base. The transition metal inorganic salt refer to inorganic salt of Ru, Rh, Pd and Ir, preferably palladium acetate. The inorganic base is preferably lithium tert butyl alcohol; the organic phosphine ligand is preferably 1,2-bis (diphenylphosphine) ethane.

In step (b), the mole ratio of the compound shown in formula (4), the oxazoline compound shown in formula (5), the transition metal inorganic salt, the organic phosphorus ligand and the inorganic base is 1:1-5: 0.01-1: 0.02-2: 2-10, preferably 1:1-3: 0.01-0.1: 0.02-0.1: 2-4.

The step (b) is carried out in an organic solvent, which is any one of benzene, carbon tetrachloride, petroleum ether, tetrahydrofuran, dimethylformamide, ether, dichloromethane, trichloromethane, toluene, xylene, cyclohexane, n-hexane, n-heptane, dioxane and acetonitrile, preferably dioxane. The reaction temperature is −0° C. to 150° C., preferably heated to reflux for reaction, and reaction time is 1 hour to 48 hours.

The invention provides an effective method for synthesizing enantiopure alkane compounds from 1,1-disubstituted olefins and atmospheric hydrogen with high enantioselectivity, using FeX₂-8-OIQ complex, especially chiral FeX₂-8-OIQ complex as catalyst, silane compound and acetonitrile as co catalyst.

Compared with the previous methods of asymmetric hydrogenation of alkenes, this method is applicable to a variety of different types of olefins. The synthesis of substrate of the olefins is very simple. The method of the present invention has mild reaction conditions, simple operation, and high atom economy. In addition, the reaction does not require the addition of any other toxic transition metal salts (such as ruthenium, rhodium, palladium, etc.), and has great practical application value in the synthesis of drugs and materials. The invention can perform selective catalytic hydrogenation of the polyolefin using a large resistance catalyst. The conversion rate of the reaction of the present invention is good, generally reaching >99%, and the enantiomer selectivity is also high, generally being 70%-99%.

### BRIEF DESCRIPTION OF DRAWINGS

FIG. 1: X-ray diffraction spectrogram of the catalyst of formula IV-2.

FIG. 2: X-ray diffraction spectrogram of the asymmetric hydrogenation product of formula II-3.

### DETAILED DESCRIPTION

The technical scheme of the invention is further described in detail by specific embodiments, but the protection scope of the invention is not limited to this.

The catalyst used in the embodiment is shown below, and the compound shown in Formula IV-1 is referred to as FeCl₂-8-OIQ for short.

The preferred amount of metal complex is 0.001-10 mol %, more preferably 0.1-5 mol %.

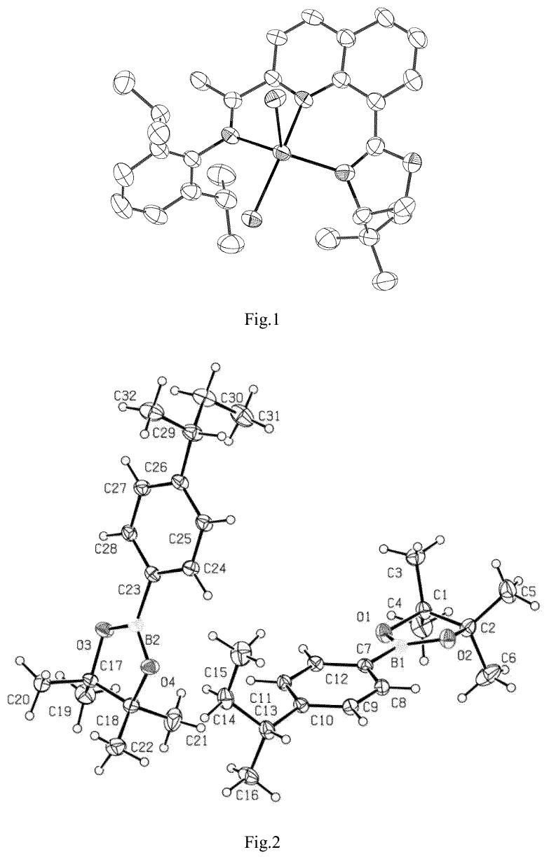

The X-ray diffraction spectrogram of Formula IV-2 is shown in FIG. 1, CCDC Number: 2011869.

The catalyst synthesis route is as follows:

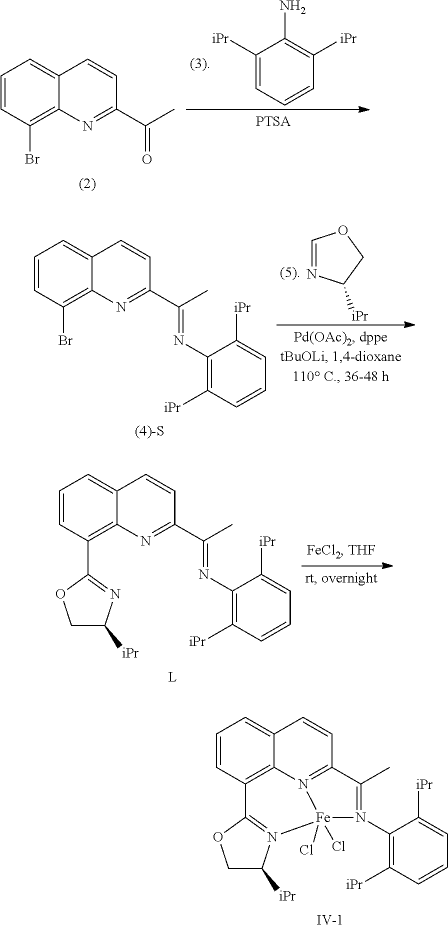

The synthesis steps of the catalyst are as follows. The amine compounds shown in Formula (3) are commercially available, and the 2-acyl-8-bromoquinoline compounds shown in Formula (2) are prepared in accordance with the literature (K. Żukowska, E. Pump, A. E. Pazio, K. Woźniak, L. Cavallo, C. Slugovc Beilstein J. Org. Chem. 2015, 11, 1458.) o Oxazoline compounds shown in Formula (5) are synthesized according to the literature (J. Chen, T. Xi, Z. Lu Org. Chem. Front., 2018, 5, 247.)

[symbol]

Preparation of 8-bromo-2-acetylquinoline (2)

Under the protection of nitrogen, the 8-bromo-formylquinoline (6.9451 g, 29.3 mmol, 1.0 equiv) and ethyl ether (147 ml) were added in three necked flask (250 mL). Then methyl magnesium bromide (3 M in hex 12.7 mL 1.3 equiv) was dropped at 0 ° C. After dropping, the reaction stirred in room temperature for 12 hours. The mixture was quenched with saturated aqueous ammonium chloride solution (10 mL). The aqueous layer was separated and extracted with ether (3 times). The organic layers were combined, dried over anhydrous Na₂SO₄, filtered and concentrated in vacuo. The crude material was concentrated and diluted with dichloromethane, 22.31 g of PDC (pyridinium dichromate 58.8 mmol, 2.0 equiv) and silica gel (23.10 g) were added in reaction and stirring overnight at room temperature. After the reaction conducted, the solid was filtered and wash with dichloromethane, concentrated in vacuo to obtain a yellow solid, The crude material was subsequently purified via column chromatography on silica gel to afford the 8-bromo-2-acetylquinoline with white solid (17.4 mmol, 4.3615 g, 59% yield).

¹H NMR (400 MHz, CDCl₃) δ 8.07-7.99 (m, 2H), 7.93 (d, J=8.0 Hz, 1H), 7.65-7.58 (m, 1H), 7.56 (d, J=8.8 Hz, 1H), 2.96 (s, 3H); the data is consistent with the literature [S. Nagy, L. N. Winslow, S. Mihan; L. Lukesova, E. Nifant'ev, P. V. Ivchenko, V. Bagrov, US Patent 2012/0016092 2012.]

Preparation of 8-bromo-2-iminoquinoline (4)-S

2,6-Diisopropylaniline (4.2516 g, 24 mmol, 1.2 equiv) [symbol] 8-Bromo-2-acetylquinoline (5.0 g, 20 mmol, 1.0 equiv) were added in toluene (50 mL), then P-toluenesulfonic acid monohydrate (0.0761 g, 0.40 mmol, 2 mol %) was added, the mixtures was refluxed for 24 h and the water was separated by water separator, then the crude product was recrystallized with ethanol to afford the (4)-S with 7.3078 g (17.9 mmol, 90% yield) [symbol] IR (neat): 2959, 2924, 1696, 1643, 1493, 1462, 1362 cm^{31 1}; 1H NMR: (400.0 MHz, CDCl3) δ 8.60 (d, J=8.8 Hz, 1H), 8.24 (d, J=8.4 Hz, 1H), 8.10 (d, J=7.2 Hz, 1H), 7.86 (d, J=8.0 Hz, 1H), 7.46 (dd, J=8.0,7.6 Hz, 1H), 7.22-7.17 (m, 2H), 7.15-7.10 (m, 1H), 2.80-2.72 (m, 2H), 2.43 (s, 3H), 1.17 (d, J=3.2 Hz, 6H), 1.15 (d, J=3.2 Hz, 6H); 13C NMR: (100.6 MHz, CDCl3) δ 167.3, 156.5, 146.5, 144.3, 136.6, 135.5, 133.1, 130.0, 127.8, 127.4, 125.9, 123.7, 123.0, 119.4, 28.3, 23.2, 22.8, 16.9; HRMS (ESI) calculated for [C23H26BrN2]+ (M+H+), requires m/z 409.1279, found m/z 409.1290.

### Synthesis of Ligand L

Under nitrogen, (4)-S (1.0213 g, 2.52 mmol, 1 equiv) and (S)-isopropyl oxazoline (0.3821 g, 3.375 mmol, 1.35 equiv) were added in dioxane (15 mL), Pd(OAc)₂ (0.0281 g, 0.0125 mmol, 5 mol %), dppe(0.0558 g, 0.14 mmol, 5.6 mol %), tBuOLi (0.4005 g, 5 mmol, 2 equiv) were added into the system, respectively. The reaction was degassed three times and stirred in 110° C. for 41 h, The crude material was subsequently purified via column chromatography on silica gel to afford the ligand L with 0.8456 g (1.9 mmol, 76% yield).

### Synthesis of Iron Catalyst:

A 50 mL Schleck flask was charged with 0.95 eq. of FeCl₂, THF (0.1 M) and a solution of 1.0 eq. of (S)-La in THF under atmosphere of nitrogen. The mixture was stirred at room temperature for 17 h. The solvent was removed in vacuo. The resulting mixture was washed with ether and filtered under air. The cake was washed with ether and dried in vacuo to afford IV-1 and IV-2.

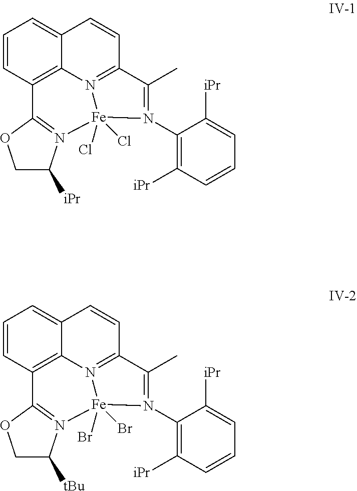

### Example 1

### the FeX₂-8-OI-Catalyzed Asymmetric Hydrogenation of 1,1-Disubstituted Alkenes

General procedure for asymmetric hydrogenation of 1,1-disubstituted alkenes: A 25 mL flame-dried Schleck flask was cooled at room temperature under nitrogen, charged with LeFeCl₂ (0.025 mmol), alkene (0.50 mmol), hydrosilane (0.10 mmol), the system was purged one time, The acetonitrile (0.10 mmol) and dry toluene (1 mL) were added successively, then NaBHEt3 (1 M in THF) (75 μL, 0.075 mmol) was injected slowly, the reaction was stirred 5 min, a balloon with hydrogen was added and the system was purged three times. The mixture was stirred for 12 hours in room temp. The reaction was quenched by PE. The mixture was filtered through a pad of silica gel and washed with PE (50 mL). The filtrate was concentrated without further purification to afford the corresponding product.

II-1: (R)-1-(sec-butyl)-4-methoxybenzene

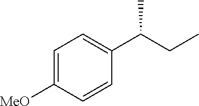

A colorless oil, >99% conversion. Optical Rotation: [α]₂₀^{D}=−23.2 (c 1.11, CHCl₃), 99% ee. ¹H NMR: (400.0 MHz, CDCl₃) δ 7.10 (d, J=8.4 Hz, 2H), 6.84 (d, J=8.4 Hz, 2H), 3.79 (s, 3H), 2.59-2.50 (m, 1H), 1.61-1.49 (m, 2H), 1.21 (d, J=7.2 Hz, 3H), 0.81 (t, J=7.6 Hz, 3H).

II-2: (R)-1-(benzyloxy)-4-(sec-butyl)benzene

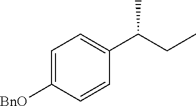 [SMILES: CC[C@@H](C)c1ccc(OCc2ccccc2)cc1]

A colorless oil, >99% conversion. Optical Rotation: [α]²⁰_{D}=−8.6 (c 1.35, CHCl₃). 98% ee. IR (neat): 2961, 2925, 1510, 1488 cm⁻¹; ¹H NMR: (400.0 MHz, CDCl₃) δ 7.45-7.30 (m, 5H), 7.10 (d, J=8.8 Hz, 2H), 6.91 (d, J=8.4 Hz, 2H), 5.04 (s, 2H), 2.59-2.51 (m, 1H), 1.60-1.52 (m, 2H), 1.21 (d, J=6.8 Hz, 3H), 0.82 (t, J=7.2 Hz, 3H); ¹³C NMR: (100.0 MHz, CDCl₃) δ 156.9, 140.1, 137.3, 128.5, 127.9, 127.8, 127.5, 114.5, 70.0, 40.8, 31.3, 22.0, 12.2; HRMS (EI) calculated for [C₁₇H₂₀O]⁺ requires m/z 240.1514, found m/z 240.1516.

II-3: (R)-2-(4-(sec-butyl)phenyl)-4,4,5,5-tetramethyl-1,3,2-dioxaborolane

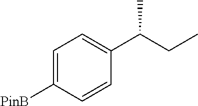

A colorless oil, >99% conversion. Optical Rotation: [α]²⁰_{D}=−2.1 (c 1.18, CHCl₃). 94.4% ee, IR (neat): 2960, 2855, 1611, 1401, 1362, 1145 cm⁻¹; ¹H NMR: (400.0 MHz, CDCl₃) δ 7.75 (d, J=8.0 Hz, 2H), 7.19 (d, J=8.0 Hz, 2H), 2.65-2.55 (m, 1H), 1.65-1.55 (m, 2H), 1.33 (s, 12H), 1.23 (d, J=6.8 Hz, 3H), 0.81 (t, J=7.6 Hz, 3H); ¹³C NMR: (100.0 MHz, CDCl₃) δ 151.1, 134.8, 126.5, 83.6, 41.9, 31.0, 24.8, 21.7, 12.2; HRMS (EI) calculated for [C₁₆H₂₅O₂B]⁺ requires m/z 260.1948, found m/z 260.1949. The x-ray diffraction was added in FIG. 2. CCDC number: 2055703.

II-4: (R)-(4-(sec-butyl)phenyl)(methyl)sulfane

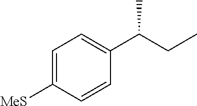

A colorless oil, >99% conversion. Optical Rotation: [α]²⁰_{D}=−18.1 (c 1.03, CHCl₃). 95.1% ee, IR (neat): 2960, 2923, 1561, 1494, 1458, 1144 cm⁻¹; ¹H NMR: (400.0 MHz, CDCl₃) δ 7.21 (d, J=8.0 Hz, 2H), 8.3 (d, J=8.0 Hz, 2H), 2.60-2.51 (m, 1H), 2.47 (s, 3H), 1.61-1.54 (m, 2H), 1.21 (d, J=6.8 Hz, 3H), 0.81 (t, J=7.6 Hz, 3H); ¹³C NMR: (100.6 MHz, CDCl₃) δ 144.9, 135.0, 127.6, 127.1, 41.2, 31.1, 21.8, 16.3, 12.2; HRMS (EI) calculated for [C₁₁H₁₆S]⁺ requires m/z 180.0973, found m/z 180.0974.

II-5: (R)-5-(sec-butyl)benzo[d][1,3]dioxole

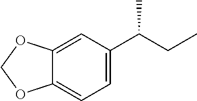

A colorless oil, >99% conversion. Optical Rotation: [α]²⁰_{D}=−12.8 (c 0.30, CHCl₃). 98.6% ee, ¹H NMR: (400.0 MHz, CDCl₃) δ 7.11-7.06 (m, 5H), 2.60-2.51 (m, 1H), 2.32 (s, 3H), 1.61-1.52 (m, 2H), 1.22 (d, J=6.8 Hz, 3H), 0.82 (t, J=7.2 Hz, 3H). IR (neat): 2961, 2927, 1487, 1440, 1249 cm⁻¹; ¹H NMR: (400.0 MHz, CDCl₃) δ 6.73 (d, J=8.0 Hz, 1H), 6.68 (s, 1H), 6.62 (d, J=8.0 Hz, 1H), 5.92 (s, 2H), 2.57-2.47 (m, 1H), 1.60-1.48 (m, 2H), 1.19 (d, J=6.8 Hz, 3H), 0.81 (t, J=7.2 Hz, 3H); ¹³C NMR: (100.0 MHz, CDCl₃) δ 147.5, 145.4, 141.7, 119.9, 107.9, 107.2, 100.7, 41.5, 31.3, 22.1, 12.2; HRMS (EI) calculated for [C₁₁ H₁₄O₂]⁺ requires m/z 178.0994, found m/z 178.0992.

II-6: (R)-1-(sec-butyl)-4-fluorobenzene

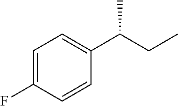

A colorless oil, >99% conversion. Optical Rotation: [α]²⁰_{D}=−12.7 (c 1.25, CHCl₃). 92.1% ee. ¹H NMR: (400.0 MHz, CDCl₃) δ 7.16-7.09 (m, 2H), 7.00-6.92 (m, 2H), 2.63-2.53 (m, 1H), 1.61-1.51 (m, 2H), 1.21 (d, J=6.8 Hz, 3H), 0.80 (t, J=7.2 Hz, 3H); ¹³C NMR: (100.6 MHz, CDCl₃) δ 161.1 (d, J=241.3 Hz), 143.2 (d, J=2.9 Hz), 128.3 (d, J=7.3 Hz), 114.9(d, J=20.4 Hz), 41.0, 31.3, 22.0, 12.1; ¹⁹F NMR: (376 MHz, CDCl₃) δ-118.1.

II-7: (R)-1-(sec-butyl)-4-chlorobenzene

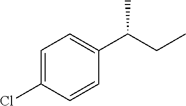

A colorless oil, >99% conversion. Optical Rotation: [α]²⁰_{D}=−3.2 (c 1.37, CHCl₃). 94.3% ee, IR (neat): 2958, 2925, 2855, 1493, 1461, 1378 cm⁻¹; ¹H NMR: (400.0 MHz, CDCl₃) δ 7.25 (d, J=8.4 Hz, 2H), 7.10 (d, J=8.4 Hz, 2H), 2.61-2.53 (m, 1H), 1.62-1.51 (m, 2H), 1.21 (d, J=6.8 Hz, 3H), 0.80 (t, J=7.2 Hz, 3H); ¹³C NMR: (100.6 MHz, CDCl₃) δ 146.1, 131.3, 128.4, 128.3, 41.1, 31.1, 21.8, 12.1; HRMS (EI) calculated for [C₁₀H₁₃Cl]⁺ requires m/z 168.0706, found m/z 168.0706.

II-8: (R)-1-(sec-butyl)-3-methoxybenzene

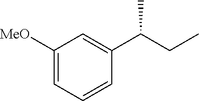

A colorless oil, >99% conversion. Optical Rotation: [α]²⁰_{D}=−21.4 (c 0.97, CHCl₃). 95.4% ee, IR (neat): 2960, 2922, 1604, 1487, 1458, 1261 cm⁻¹; ¹H NMR: (400.0 MHz, CDCl₃) δ 7.21 (t, J=7.8 Hz, 1H), 6.79-6.72 (m, 3H), 3.80 (s, 3H), 2.61-2.52 (m, 1H), 1.64-1.00 (m, 2H), 1.23 (d, J=6.8 Hz, 3H), 0.83 (t, J=7.2 Hz, 3H); ¹³C NMR: (100.0 MHz, CDCl₃) δ 159.6, 149.5, 129.1, 119.5, 113.0, 110.7, 55.1, 41.7, 31.1, 21.8, 12.2; HRMS (EI) calculated for [C₁₁H₁₆O]⁺ requires m/z 164.1201, found m/z 164.1203.

II-9: (R)-1-(sec-butyl)-2-methoxybenzene

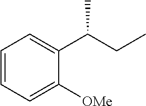

A colorless oil, >99% conversion. Optical Rotation: [α]²⁰_{D}=−1.4 (c 0.51, CHCl₃). 77.4% ee, IR (neat): 2921, 2853, 1511, 1464, 1380, 1246 cm⁻¹; ¹H NMR: (400.0 MHz, CDCl₃) δ 7.19-7.12 (m, 2H), 6.95-6.82 (m, 2H), 3.81 (s, 3H), 3.16-3.04 (m, 1H), 1.70-1.54 (m, 2H), 1.19 (d, J=6.8 Hz, 3H), 0.84 (t, J=6.8 Hz, 3H); ¹³C NMR: (100.6 MHz, CDCl₃) δ 157.1, 135.9, 126.7, 126.4, 120.5, 110.4, 55.3, 33.4, 29.8, 20.4, 12.1; HRMS (EI) calculated for [C₁₁H₁₆O]⁺ requires m/z 164.1201, found m/z 164.1202.

II-10: (R)-5-(sec-butyl)-1-methyl-1H-indole

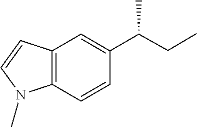

A colorless oil, >99% conversion. Optical Rotation: [α]²⁰_{D}=−16.0 (c 1.11, CHCl₃). 97.9% ee, IR (neat): 2958, 2919, 1513, 1490 1450, 1352 cm⁻¹; ¹H NMR: (400.0 MHz, CDCl₃) δ 7.42 (s, 1H), 7.28-7.23 (m, 1H), 7.07 (d, J=7.6 Hz, 1H), 7.01 (d, J=2.8 Hz, 1H), 6.42 (d, J=2.8 Hz, 1H), 3.77 (s, 3H), 2.74-2.63 (m, 1H), 1.71-1.59 (m, 2H), 1.29 (d, J=6.8 Hz, 3H), 0.83 (t, J=7.2 Hz, 3H); ¹³C NMR: (100.0 MHz, CDCl₃) δ 138.6, 135.4, 128.7, 128.5, 121.1, 118.6, 108.8, 100.5, 41.7, 32.7, 31.6, 22.6, 12.4; HRMS (EI) calculated for [C₁₃H₁₇N]⁺ requires m/z 187.1361, found m/z 187.1362.

II-11: (R)-1-(hexan-2-yl)-4-methoxybenzene

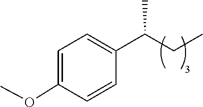

A colorless oil, >99% conversion. Optical Rotation: [α]²⁰_{D}=−27.4 (c 0.89, CHCl₃). 93.9% ee, IR (neat): 2957, 2927, 1611, 1512, 1461, 1247 cm⁻¹; ¹H NMR: (400.0 MHz, CDCl₃) δ 7.10 (d, J=8.8 Hz, 2H), 6.83 (d, J=8.8 Hz, 2H), 3.79 (s, 3H), 2.67-2.57 (s, 1H), 1.56-1.48 (m, 2H), 1.34-1.07 (m, 7H), 0.85 (t, J=6.8 Hz, 3H); ¹³C NMR: (100.0 MHz, CDCl₃) δ 157.6, 140.1, 127.8, 113.6, 55.2, 39.0, 38.3, 29.9, 22.8, 22.5, 14.0; HRMS (EI) calculated for [C₁₃H₂₀O]⁺ requires m/z 192.1514, found m/z 192.1514.

II-12: (R)-1-(heptan-2-yl)-4-methoxybenzene

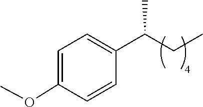

A colorless oil, >99% conversion. Optical Rotation: [α]²⁰_{D}=−26.7 (c 0.75, CHCl₃). 94.8% ee, IR (neat): 2923, 2854, 1612, 1512, 1461, 1375 cm⁻¹; ¹H NMR: (400.0 MHz, CDCl₃) δ 7.09 (d, J=8.8 Hz, 2H), 6.83 (d, J=8.4 Hz, 2H), 3.79 (s, 3H), 2.67-2.57 (m, 1H), 1.55-1.47 (m, 2H), 1.30-1.10 (m, 9H), 0.85 (t, J=6.4 Hz, 3H); ¹³C NMR: (100.0 MHz, CDCl₃) δ 157.6, 140.1, 127.7, 113.6, 55.1, 39.0, 38.6, 31.9, 27.4, 22.6, 22.5, 14.1; HRMS (EI) calculated for [C₁₄H₂₂O]⁺ requires m/z 206.1671, found m/z 206.1672.

II-13: (R)-1-methoxy-4-(octan-2-yl)benzene

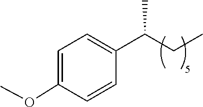

A colorless oil, >99% conversion. Optical Rotation: [α]²⁰_{D}=−23.4 (c 1.20, CHCl₃). 90.8% ee, IR (neat): 2955, 2854, 1612 1512, 1461, 1375 cm⁻¹; ¹H NMR: (400.0 MHz, CDCl₃) δ 7.09 (d, J=8.8 Hz, 2H), 6.83 (d, J=8.4 Hz, 2H), 3.79 (s, 3H), 2.67-2.57 (m, 1H), 1.56-1.48 (m, 2H), 1.30-1.10 (m, 11H), 0.85 (t, J=6.4 Hz, 3H); ¹³C NMR: (100.0 MHz, CDCl₃) δ 157.6, 140.1, 127.7, 113.6, 55.1, 39.1, 38.6, 31.8, 29.4, 27.7, 22.6, 22.5, 14.1; HRMS (EI) calculated for [C₁₅H₂₄O]⁺ requires m/z 220.1827, found m/z 220.1826.

II-14: (R)-N,N-dimethyl-4-(p-tolyl)pentan-1-amine

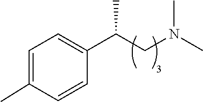

A colorless oil, >99% conversion. Optical Rotation: [α]²⁰_{D}=−17.4 (c 0.92, CHCl₃). 96.6% ee, IR (neat): 2941, 2858, 2763, 1651, 1515, 1374 cm⁻¹; ¹H NMR: (400.0 MHz, CDCl₃) δ 7.12-7.04 (m, 4H), 2.70-2.59 (m, 1H), 2.31 (s, 3H), 2.25-2.14 (m, 8H), 1.61-1.52 (m, 2H), 1.47-1.28 (m, 2H), 1.23 (d, J=6.8 Hz, 3H); ¹³C NMR: (100.0 MHz, CDCl₃) δ 144.4, 135.2, 128.9, 126.8, 59.8, 45.4, 39.5, 36.1, 25.8, 22.4, 20.9; HRMS (EI) calculated for [C₁₄H₂₃N]⁺ requires m/z 205.1830, found m/z 205.1832.

II-15: (R)-propane-1,2-diyldibenzene

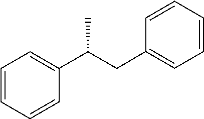

A colorless oil, >99% conversion. Optical Rotation: [α]²⁰_{D}=−33.9 (c 1.09, CHCl₃). 84.3% ee, ¹H NMR: (400.0 MHz, CDCl₃) δ 7.35-7.01 (m, 10H), 3.07-2.88 (m, 2H), 2.83-2.69 (m, 1H), 1.24 (d, J=6.8 Hz, 3H); ¹³C NMR: (100.0 MHz, CDCl₃) δ 147.0, 140.8, 129.1, 128.3, 128.1, 127.0, 126.0, 125.8, 45.0, 41.8, 21.1.

II-16: (1R,3R)-3-(4-((R)-sec-butyl)phenoxy)-1,7,7-trimethylbicyclo[2.2.1]heptane

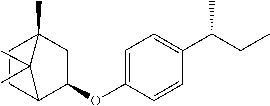 [SMILES: CC[C@@H](C)c1ccc(O[C@@H]2C[C@@]3(C)CCC2C3(C)C)cc1]

A colorless oil, >99% conversion. Optical Rotation: [α]²⁰_{D}=−88.3 (c 0.79, CHCl₃). 96% ee, IR (neat): 2955, 2875, 1610, 1510, 1455, 1245 cm⁻¹; ¹H NMR: (400.0 MHz, CDCl₃) δ 7.06 (d, J=8.8 Hz, 2H), 6.76 (d, J=8.8 Hz, 2H), 4.32-4.25 (m, 1H), 2.58-2.47 (m, 1H), 2.40-2.30 (m, 1H), 2.29-2.18 (m, 1H), 1.81-1.69 (m, 2H), 1.61-1.50 (m, 2H), 1.23-1.37 (m, 2H), 1.20 (d, J=7.2 Hz, 3H) 1.13 (dd, J=13.2, 3.2 Hz, 1H), 0.97-0.88 (m, 9H), 0.81 (t, J=7.2 Hz, 3H); ¹³C NMR: (100.0 MHz, CDCl₃) δ 157.2, 139.2, 127.7, 115.1, 82.7, 49.5, 47.5, 45.2, 40.8, 36.9, 31.3, 27.9, 26.8, 22.0, 19.7, 19.0, 13.8, 12.3. HRMS (EI) calculated for [C₂₀H₃₀O]⁺ requires m/z 286.2297, found m/z 286.2297.

II-17: (R,E)-1-(sec-butyl)-4-((3,7-dimethylocta-2,6-dien-1-yl)oxy)benzene

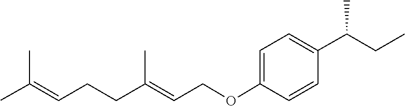 [SMILES: CC[C@@H](C)c1ccc(OC/C=C(\C)CCC=C(C)C)cc1]

A colorless oil, >99% conversion. Optical Rotation: [α]²⁰_{D}=−13.1 (c 0.78, CHCl₃). 97.2% ee, IR (neat): 2963, 2922, 1671, 1611, 1511, 1455 cm⁻¹; ¹H NMR: (400.0 MHz, CDCl₃) δ 7.08 (d, J=8.8 Hz, 2H), 6.85 (d, J=8.4 Hz, 2H), 5.50 (t, J=6.8 Hz, 1H), 5.10 (t, J=5.2 Hz, 1H), 4.51 (d, J=6.8 Hz, 2H), 2.59-2.48 (m, 1H), 2.18-2.04 (m, 4H), 1.73(s, 3H),1.68 (s, 3H), 1.58 (s, 3H), 1.57-1.51 (m, 2H), 1.21 (d, J=6.8 Hz, 3H), 0.81 (t, J=7.6 Hz, 3H); ¹³C NMR: (100.0 MHz, CDCl₃) δ 157.0, 140.8, 139.6, 131.7, 127.8, 123.8, 119.8, 114.4, 64.8, 40.8, 39.5, 31.3, 26.3, 25.6, 22.0, 17.6, 16.6, 12.2. HRMS (EI) calculated for [C₂₀H₃₀O]⁺ requires m/z 286.2297, found m/z 286.2298.

II-18: (R)-2-(3-(3-methoxy-4-methylphenyl)butyl)-1,3-dioxolane

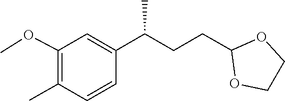 [SMILES: COc1cc([C@H](C)CCC2OCCO2)ccc1C]

A colorless oil, >99% conversion. Optical Rotation: [α]^{°}_{D}=−11.4 (c 1.98, CHCl₃). 97.2% ee, IR (neat): 2954, 2926, 1611, 1582, 1462, 1256 cm⁻¹; ¹H NMR: (400.0 MHz, CDCl₃) δ 7.03 (d, J=7.6 Hz, 1H), 6.71-6.63 (m, 2H), 4.81 (t, J=4.8 Hz, 1H), 4.01-3.79 (m, 7H), 2.71-2.59 (m, 1H), 2.17 (s, 3H), 1.73-1.49 (m, 4H), 1.25 (d, J=7.2 Hz, 3H); 13C NMR: (100.0 MHz, CDCl3) δ 157.6, 146.1, 130.4, 124.0, 118.6, 108.8, 104.6, 64.8, 55.2, 39.9, 32.4, 32.1, 22.4, 15.8; HRMS (EI) calculated for [C₁₅H₂₂O₃]+ requires m/z 250.1569, found m/z 250.1569.

II-19: (R)-5-methyl-6,7,8,9-tetrahydro-5H-benzo[7]annulene

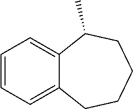

A colorless oil, >99% conversion. Optical Rotation: [α]²⁰_{D}=+10.6 (c 0.50, CHCl₃). 90.5% ee, ¹H NMR: (400.0 MHz, CDCl₃) δ 7.22-7.04 (m, 4H), 3.10-2.99 (m, 1H), 2.93-2.83 (m, 1H), 2.83-2.74 (m, 1H), 1.98-1.86 (m, 1H), 1.86-1.68 (m, 3H), 1.51-1.30 (m, 5H); ¹³C NMR: (100.0 MHz, CDCl₃) δ 146.5, 142.8, 129.2, 126.0, 125.6, 125.2, 37.7, 36.1, 36.0, 30.1, 27.8, 20.4.

II-20: (R) -1-(3,4-dimethylpentyl)-4-methoxybenzene

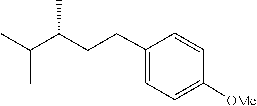

A colorless oil, 42% conversion. 54% ee. ¹ H NMR: (400.0 MHz, CDCl₃) δ 7.10 (d, J=8.0 Hz, 2H), 6.82 (d, J=8.0 Hz, 2H), 3.79 (s, 3H), 2.66-2.55 (m, 1H), 2.53-2.41 (m, 1H), 1.69-1.56 (m, 2H), 1.44-1.25 (m, 2H), 0.93-0.77 (m, 9H).

### Example 2

### Selective Hydrogenation of 1,1-Disubstituted Olefins Using the Catalyst FeCl₂-8-OIQ (tBuoxazole Ring) with Large Steric Hindrance

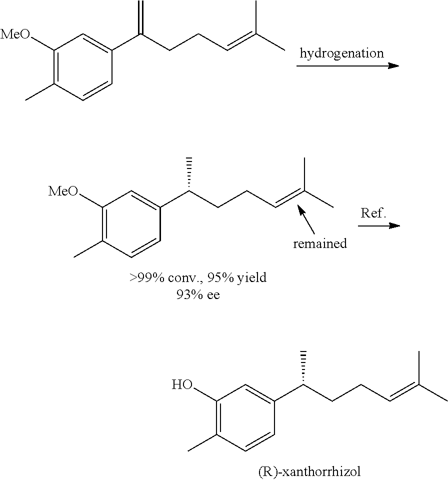

A 25 mL flame-dried Schleck flask was cooled at room temperature under nitrogen, charged with LeFeCl₂ (0.025 mmol), alkene (0.50 mmol), hydrosilane (0.10 mmol), the system was purged one time, The acetonitrile (0.10 mmol) and dry toluene (1 mL) were added successively, then NaBHEt3 (1 M in THF) (75 μL, 0.075 mmol) was injected slowly, the reaction was stirred 5 min, a balloon with hydrogen was added and the system was purged three times. The mixture was stirred for 12 hours in room temp. The reaction was quenched by PE. The mixture was filtered through a pad of silica gel and washed with PE (50 mL). The filtrate was concentrated without further purification to afford the corresponding product with >99% conversion. Optical Rotation: [α]²⁰_{D}=−32.5 (c 1.75, CHCl₃). 93% ee. The (R)-xanthorrhizol could be synthesized according to the literature.

### Example 3

### Synthesis of Drug Intermediates

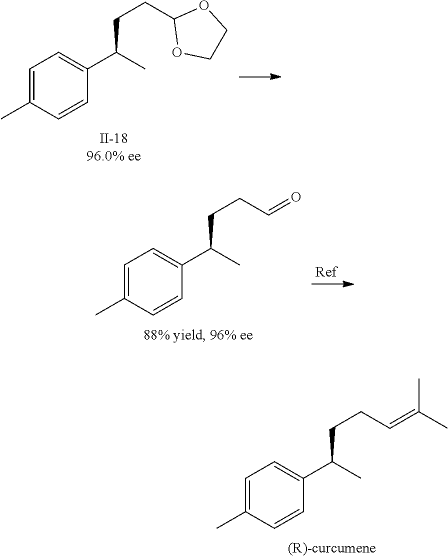

To (R)-2-(3-(p-tolyl)butyl)-1,3-dioxolane (II-18) (0.0404 g, 1 equiv) was added a solution of AcOH (6 ml) and H₂O (2 ml). The reaction mixture was stirred at 60° C. for 5 h and then was cooled down to room temperature. Adding the sodium hydroxide solution to adjust pH to 7, then exacted with ethyl acetate (3×10 mL), the solvent was removed in vacuo, providing the crude residue. The crude material was purified by column chromatography (PE/EA (30/1)) to give the intermediate of (R)-curcumene (0.0286 g, 88% yield) as a colorless oil. Optical Rotation: [α]²⁰_{D}=−33.9 (c 0.91, CHCl₃). 96% ee. Optical Rotation: [α]²⁰_{D}=−33.9 (c 0.91, CHCl₃). 96% ee, ¹H NMR: (400.0 MHz, CDCl₃) δ 9.68 (t, J=1.6 Hz, 1H), 7.14-7.03 (m, 4H), 2.75-2.63 (m, 1H), 2.39-2.24 (m, 2H), 1.99-1.79 (m, 2H), 1.26 (d, J=7.2 Hz, 3H); ¹³C NMR: (100.6 MHz, CDCl₃) δ 202.4, 142.9, 135.7, 129.1, 126.8, 42.1, 38.8, 30.3, 22.3, 20.9. (: Song, S.; Zhu, S. -F.; Yang, S.; Li, S.; Zhou, Q. -L. Angew. Chem. Int. Ed. 2012, 51, 2708.)

### Example 4

### Comparative Experiment on Catalytic Performance and Reaction Conditions of Various Catalysts

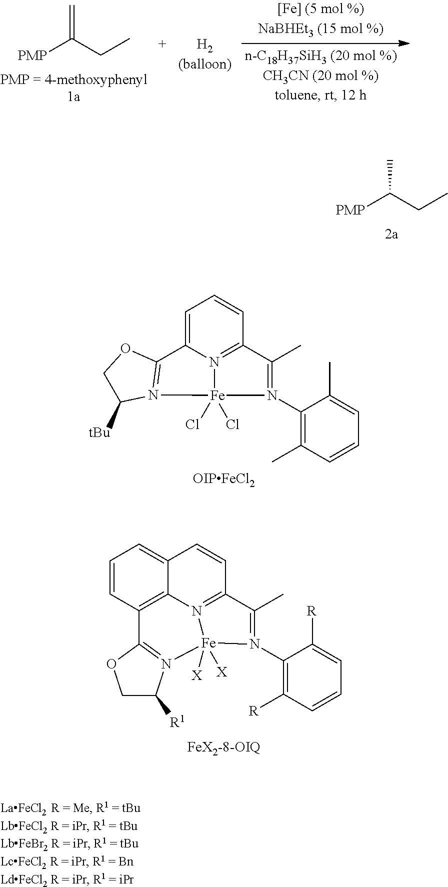

| | | yield of | recovery | isom. | ee of 2a | |
|---|---|---|---|---|---|---|
| entry | cat. | 2a (%)^{b} | of 1a (%) | (%) | (%)^{c} | Note |
| 1 | OIP•FeCl₂ | 25 | 51 | 24 | 58 | LP17107 |
| 2 | La•FeCl₂ | >99 | 0 | 0 | 88 | LP15115-B |
| 3 | Lb•FeCl₂ | 98 | 0 | 2 | 99 | LP15114-A |
| 4 | Lc•FeCl₂ | 98 | 0 | 2 | 97 | LP15115-A |
| 5 | Ld•FeCl₂ | >99 | 0 | 0 | 99 | LP17005 |

^{a} The reactions were conducted using alkene (0.5 mmol), H₂ balloon, hydrosilane (20 mol %), CH₃CN (20 mol %), iron cat. (5 mol %), NaBHEt₃ (15 mol %), and toluene (1 mL) at rt for 12 h;
^{b}The conversions and recoveries were determined by ¹H NMR using TMSPh as an internal standard; ee values were determined by GC using chiral column.

1. A method for preparing chiral alkyl compounds by asymmetric hydrogenation of olefins catalyzed by iron complex catalyst, wherein the method is as follows: a disubstituted olefin shown in Formula I is used as a raw material, atmospheric hydrogen is used as hydrogen source, FeX₂-8-OIQ complex is used as catalyst, hydrosilane and acetonitrile are used as co catalysts, and a chiral alkyl compound shown in Formula II is prepared by reaction for 12-24 hours under the activation of a reducing agent; 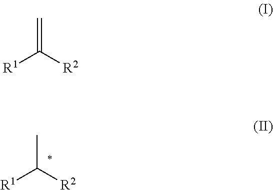
in Formula II, * represents a chiral carbon atom;
in Formula I or Formula II, R¹ is C₂˜C₈ alkyl, naphthyl, a group shown in Formula III, or a N and O containing heterocyclic aryl group of C₄˜C₁₀;
in R¹, the H on C₂˜C₈ alkyl is not substituted or substituted by at least one substituent A, and the substituent A is phenyl, naphthyl, heterocyclic aryl or substituted phenyl; heterocyclic aryl is indolyl, pyridinyl, pyrrolyl, thiophenyl or furanyl; substituted phenyl is the phenyl group in which H on phenyl is replaced by at least one substituent B, and the substituent B is C₁˜C₃ alkylp, C₁˜C₃ alkoxy, halogen or C₁˜C₃ alkylthio;
in R¹, the N and O containing heterocyclic aryl group of C₄˜C₁₀ is pyridinyl, pyrrolyl, indolyl, benzodioxazolyl, benzoxazolyl or furanyl;
in R¹, the H on naphthyl and the N and O containing heterocyclic aryl group of C₄˜C₁₀ are not substituted or substituted by at least one substituent C, and the substituent C is C₁˜C₃ alkyl or C₁˜C₃ alkoxy; 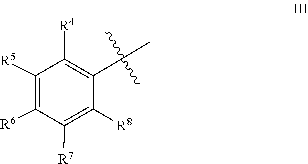
in R¹, among the groups shown in Formula III, R⁴, R⁵, R⁶, R⁷, R⁸ are selected from any one of H, halogen, C₁˜C₃ alkyl, C₁˜C₃ alkoxy, benzyloxy, C₁˜C₃ alkylthio, tert butyl dimethyl siloxy, trifluoromethyl, dimethylamino, pinacol borate, d-borneoxy, citronellol oxy, menthol oxy or geraniol oxy, and when R⁴, R⁵, R⁶, R⁷, R⁸ are all H, Formula III is phenyl; and halogen is F or Cl;
in Formula I or Formula II, R² is C₁˜C₈ alkyl, C₂˜C₈ alkenyl, phenyl or benzyl; the H on C₁˜C₈ alkyl and C₂˜C₈ alkenyl are not substituted or substituted by at least one substituent D, which is phenyl, substituted phenyl, C₁˜C₃ amino or 1,3-dioxolacyl;
or in Formula I or Formula II, R¹ and R² are connected into a ring to form C₉˜C₁₂ benzocycloalkyl; H on C₉˜C₁₂ benzocycloalkyl is not substituted or substituted by at least one substituent E, and the substituent E is C₁˜C₃ alkyl, C₁˜C₃ alkoxy or halogen;
R¹ and R² are different substituents.

2. The method according to claim 1, wherein R¹ is C₂˜C₈ alkyl, naphthyl, 6-methoxynaphthalyl, pyridinyl, 2-methoxypyridyl, indolyl, N-methylindolyl, benzodioxazolyl or the group shown in Formula III;
in R¹, when R¹ is C₂˜C₈ alkyl, H on C₂˜C₈ alkyl is replaced by a substituent A, R¹ is expressed as R^{A}—(CH₂)ₙ—, wherein n is an integer of 2 to 8, R^{A} is a substituent A on the carbon chain, and R^{A} is phenyl, naphthyl or p-methoxyphenyl;
the group shown in Formula III is phenyl or substituted phenyl with 1-2 substituents, and the substituent on the substituted phenyl is halogen, C₁˜C₂ alkyl, C₁˜C₃ alkoxy, benzyloxy, C₁˜C₃ alkylthio, tert-butyl dimethyl siloxy, trifluoromethyl, dimethylamino, pinacol borate, d-borneol, citronellol-oxyl, menthol-oxyl or geraniol-oxyl;
R² is C₂˜C₆ alkyl, C₂˜C₆ alkenyl, phenyl or benzyl; H on the C₂˜C₆ alkyl is not substituted or substituted by substituent D, R² is C₂˜C₆ alkyl, and when H on the alkyl is substituted by substituent D, R² is expressed as R^{D}—(CH₂)ₘ—, m is an integer of 2 to 6, and the substituent D is phenyl, C₁˜C₃ amino, 4-methoxyphenyl or 1,3-dioxolacyl.

3. The method according to claim 1, wherein the catalyst FeX₂-8-OIQ complex is an optically pure compound shown in Formula IV or its enantiomer or racemate; 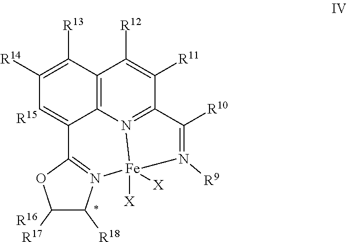
in Formula IV, R⁹ is C₁˜C₁₂ alkyl which is unsubstituted or substituted by 1 or 2 C₁˜C₄ alkoxy, C₅˜C₁₂ cycloalkyl which is unsubstituted or substituted by 1-3 substituents a, or aryl a which is unsubstituted or substituted by 1-4 substituents b; the aryl a is benzyl, phenyl or naphthyl; the substituent a is C₁˜C₄ alkyl or C₁˜C₄ alkoxy; and the substituent b is C₁˜C₄ alkyl, C₁˜C₄ alkoxy, C₁˜C₄ fluoroalkyl, C₁˜C₄ fluoroalkoxy, F or Cl;
R¹⁰ is H, C₁˜C₁₂ alkyl which is unsubstituted or substituted by 1-2 C₁˜C₄ alkoxy, C₅˜C₁₂ cycloalkyl which is unsubstituted or substituted by 1-3 substituents a, or aryl b which is unsubstituted or substituted by 1-3 substituents b; the aryl b is phenyl or naphthyl; the substituent a is C₁˜C₄ alkyl or C₁˜C₄ alkoxy; and the substituent b is C₁˜C₄ alkyl, C₁˜C₄ alkoxy, C₁˜C₄ fluoroalkyl, C₁˜C₄ fluoroalkoxy, F or Cl;
R¹¹; R¹², R¹³, R¹⁴ and R¹⁵ are independently H, C₁˜C₁₂ alkyl, C₁˜C₄ fluoroalkoxy, F, Cl, nitro or C₅˜C₁₂ cycloalkyl which is unsubstituted or substituted by 1-3 substituents a;
R¹⁶ and R¹⁷ are independently H, C₁˜C₁₂ alkyl which is unsubstituted or substituted by 1-2 C₁˜C₄ alkoxy, C₅˜C₁₂ cycloalkyl which is unsubstituted or substituted by 1-3 substituents a, or aryl a which is unsubstituted or substituted by 1-3 substituents b;
R¹⁸ is a C₁˜C₁₂ alkyl group that is not substituted or substituted by 1-2 C₁˜C₄ alkoxy, C₅˜C₁₂ cycloalkyl which is not substituted or substituted by 1-3 substituents a, or aryl a which is not substituted or substituted by 1-3 substituents b;
in Formula IV, * represents a chiral carbon atom;
X is F, Cl, Br, I, OAc or CF₃SO₃.

4. The method according to claim 3, wherein the catalyst FeX₂-8-OIQ complex is a compound shown in Formula IV, wherein R¹¹, R¹², R¹³, R¹⁴, R¹⁵, R¹⁶ and R¹⁷ are H; R¹⁰ is C₁˜C₄ alkyl or diphenylmethylene; R⁹ is C₁˜C₄ alkyl, benzyl, phenyl or 2,6-diisopropylphenyl; R¹⁸ is C₁˜C₄ alkyl, benzyl or phenyl; X is Cl or Br.

5. The method according to claim 3, wherein the FeX₂-8-OIQ complex is shown in Formula IV-1 or Formula IV-2,

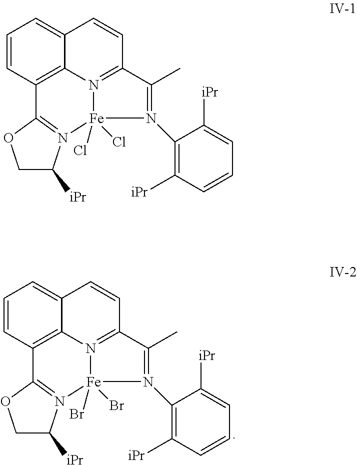

6. The method according to claim 1, wherein the silane compound is benzene silane or n-octadecyl silane.

7. The method according to claim 1, wherein an organic solvent is added to the method, and the organic solvent is any one of benzene, carbon tetrachloride, toluene, tetrahydrofuran, ether, dichloromethane, acetonitrile, dioxane, petroleum ether, cyclohexane, n-hexane, ethyl acetate, chloroform, and dimethylformamide.

8. The method according to claim 1, wherein the reducing agent is any one of sodium triethylborohydride, sodium tri-sec-butyl-borohydride, lithium triethylborohydride, sodium tert-butoxide, potassium tert-butoxide, lithium tert-butoxide, sodium tert-pentoxide, sodium ethoxide, sodium methoxide and potassium methoxide.

9. The method according to claim 1, wherein the mole ratio of the disubstituted olefin shown in Formula I, FeX₂-8-OIQ complex, silane compound, acetonitrile and reducing agent is 1:0.00001-0.1:0.02-0.2:0.1-0.3:0.06-0.3.

10. The method according to claim 1, wherein the reaction temperature is 0° C.˜room temperature.
